(19) United States
(12) Patent Application Publication
(10) Pub. No.: US 2021/0401912 A1
(43) Pub. Date: Dec. 30, 2021

(54) USE OF ANTRODIA CINNAMOMEA COMPOSITION FOR IMMUNE MODULATION

(71) Applicant: Taiwan Leader Biotech Corp., Taipei City (TW)

(72) Inventors: Chin-Chung LIN, Taipei City (TW); Tzung-Hsien LAI, Taipei City (TW); Wan-Jing CHEN, Taipei City (TW); Jent-Turn LEE, Taipei City (TW)

(21) Appl. No.: 17/003,396

(22) Filed: Aug. 26, 2020

(30) Foreign Application Priority Data
Jun. 30, 2020 (TW) 109122047

(51) Int. Cl. A61K 36/06

(52) U.S. Cl. CPC A61K 36/06

(57) ABSTRACT

A method for modulating an immune system in a subject in need thereof, comprising: administering to a subject an effective amount of a pharmaceutical composition comprising an Antrodia cinnamomea composition, wherein the Antrodia cinnamomea composition comprises: 50 wt % to 100 wt % of Antrodia cinnamomea mycelium; and 0 wt % to 50 wt % of an extract of fruiting body of Antrodia cinnamomea.

### CROSS REFERENCE TO RELATED APPLICATION

This application claims the benefits of the Taiwan Patent Application Serial Number 109122047, filed on Jun. 30, 2020, the subject matter of which is incorporated herein by reference.

### BACKGROUND

### 1. Field

The present disclosure relates to a novel application of the Antrodia cinnamomea composition for immune modulation.

### 2. Description of Related Art

The therapeutic potential of polysaccharide and triterpene derivatives is largely discussed in the field of immunological research. Antrodia cinnamomea (AC) is found in Taiwan, and it is commonly used for the treatment of inflammatory responses, infections, and cancers. For example, the Antrodia cinnamomea extract is proven to inhibit the secretion of interleukin-1β (IL-1β) and interleukin-18 (IL-18), reduce expression of the inflammasome component NLRP3, and inhibit ATP-induced ROS production and caspase-1 activation; thus, the Antrodia cinnamomea extract can be used in the treatment of inflammatory responses (Journal of Ethnopharmacology, Volume 155, Issue 1, 8 Aug. 2014, Pages 154-164). In addition, it is found that high levels of interferon-γ (IFN-γ) and tumor necrosis factor-α (TNF-α) mRNA can be detected in mice after oral Antrodia cinnamomea polysaccharide administration; the number of splenic dendritic cells, macrophages, and the surface expression of CD8α⁺ and major histocompatibility class II I-A/I-E on dendritic cells were increased; the CD4⁺/CD8⁺ ratio and number of B cells among splenocytes were also augmented; and the infection in mice also inhibited after Antrodia cinnamomea polysaccharide administration (International Immunopharmacology, Volume 8, Issue 3, March 2008, Pages 458-467). Furthermore, it is also found that the Antrodia cinnamomea extract can inhibit lung tumor growth and metastasis without affecting serum biological parameters or body weight; thus, the Antrodia cinnamomea extract is considered as a candidate for the treatment of lung cancer (Scientific Reports, Volume 9, Article number: 5145 (2019)).

The mechanism of antigen-presentation had been stressed on the interaction between dendritic cells, B cells, and MHC class II molecules. However, there are growing evidence proving that monocytes possess new functional roles in adaptive immunity, such as their emerging roles in antigen presentation, and promotion of Treg cell proliferation. Treg cells are T cells that help maintain tolerance to self-antigens, and down-regulation of effector T cells. Hence, investigating MHC-II positive monocytes provides a deep insight into immunological events.

In modern immunotherapy research, the PD1 (programmed death 1)/PD-L1 (PD-1 ligand) axis is regarded as important markers. PD1 receptor is expressed on the surfaces of activated T cells. The Programmed death-ligand 1 receptor is overexpressed in tumor cells. The PD1 receptor on T cells usually binds to the receptor of PD-L1 of tumor cells, which leads to the inhibition of the cytotoxic T cells. PD-1/PD-L1 axis is a novel target frequently described in modern immunology.

Therefore, it is desirable to provide a novel application of the Antrodia cinnamomea composition for immune modulation, which may be effective on the immunological research and the immunotherapy research.

### SUMMARY

An object of the present disclosure is to provide a novel application of the Antrodia cinnamomea composition for immune modulation.

The Antrodia cinnamomea composition of the present disclosure comprises: 50 wt % to 100 wt % of Antrodia cinnamomea mycelium; and 0 wt % to 50 wt % of an extract of fruiting body of Antrodia cinnamomea. In one example of the present disclosure, the Antrodia cinnamomea composition comprises: 50 wt % to 99 wt % of Antrodia cinnamomea mycelium; and 1 wt % to 50 wt % of an extract of fruiting body of Antrodia cinnamomea. In another example of the present disclosure, the Antrodia cinnamomea composition is consisted of: 50 wt % to 100 wt % of Antrodia cinnamomea mycelium; and 0 wt % to 50 wt % of an extract of fruiting body of Antrodia cinnamomea. In further another example of the present disclosure, the Antrodia cinnamomea composition is consisted of: 50 wt % to 99 wt % of Antrodia cinnamomea mycelium; and 1 wt % to 50 wt % of an extract of fruiting body of Antrodia cinnamomea.

When the Antrodia cinnamomea composition of the present disclosure is administered, the purpose of immune modulation can be achieved. Thus, the present disclosure provides a use of the aforesaid Antrodia cinnamomea composition to prepare a pharmaceutical composition for immune modulation. In addition, the present disclosure further provides a method for modulating an immune system in a subject in need thereof, comprising: administering to a subject an effective amount of a pharmaceutical composition comprising the aforesaid Antrodia cinnamomea composition to modulate the immune system in the subject. Herein, the subject can be human or other mammalian.

In the present disclosure, the Antrodia cinnamomea mycelium can be solid-state cultivated Antrodia cinnamomea mycelium. For example, the Antrodia cinnamomea mycelium can be solid cultured with edible whole grains, and processed by drying and grinding after harvest.

In the present disclosure, the extract of the fruiting body of Antrodia cinnamomea can be an extract using water and ethanol. For example, the extract of the fruiting body of Antrodia cinnamomea can be an ethanol extract of the fruiting body of Antrodia cinnamomea extracted with 50 wt % to 95 wt % of ethanol.

In the present disclosure, the active ingredients comprised in each grain of the Antrodia cinnamomea composition include: 4,7-dimethoxy-5-methyl-1,3-benzodioxole (2.9 mg˜17 mg), 4-hydroxy-2,3-dimethoxy-6-methyl-5-[3,7,11-trimethyl-2,6,10-dodecatriene]-2-cyclohexenone (0.35 mg˜21 mg), coenzyme Q3 (0.23 mg˜10 mg), Safrole C (0.24 mg˜9 mg), Zhankuic acid B (0.09 mg˜5 mg), Zhankuic acid H (0.25 mg˜13 mg), Zhankuic acid K (0.15 mg˜8 mg), Zhankuic acid A (0.005 mg˜2 mg), Zhankuic acid C (0.03 mg˜2 mg), dehydrosulfurenic acid (0.14 mg˜11 mg) and dehydroeburicoic acid (0.15 mg˜15 mg).

In the present embodiment, the effect of the immune modulation includes: reducing PD-L1⁺ B cells, reducing PD-L1⁺ monocytes, reducing MHC II⁺ dendritic cells, increasing effector CD4 αβ T cells and/or increasing effector CD8 αβ T cells. In particular, compared with the peripheral blood of the subject before the Antrodia cinnamomea composition or the pharmaceutical composition comprising the same is administered, decreased PD-L1⁺ B cells, decreased PD-L1⁺ monocytes, decreased MHC II⁺ dendritic cells, increased effector CD4 αβ T cells and/or increased effector CD8 αβ T cells in the peripheral blood of the subject can be observed after the Antrodia cinnamomea composition or the pharmaceutical composition comprising the same is administered. Herein, the PD-L1⁺ B cells, PD-L1⁺ monocytes, MHC II⁺ dendritic cells, effector CD4 αβ T cells and/or effector CD8 αβ T cells in the peripheral blood can be detected by flow cytometry, Enzyme-linked Immunospot Assay (ELISPOT) or immunohistochemistry (IHC).

In the present disclosure, the Antrodia cinnamomea composition or the pharmaceutical composition can be administered to a subject orally, parenterally, by inhalation spray, topically, rectally, nasally, buccally, or via an implanted reservoir.

In addition, the pharmaceutical composition of the present disclosure may comprise 300 mg to 1500 mg of the Antrodia cinnamomea composition, for example, 300 mg to 1400 mg, 300 mg to 1300 mg, 400 mg to 1200 mg, 400 mg to 1100 mg, 500 mg to 1100 mg, 500 mg to 1000 mg or 800 mg to 1000 mg of the Antrodia cinnamomea composition. Furthermore, in the pharmaceutical composition of the present disclosure, the pharmaceutical composition can be formulated into solutions, capsules, tablets, powders, spray, syrup or other suitable formulation. When the pharmaceutical composition is formulated into capsules or tablets, the aforesaid dosage of the Antrodia cinnamomea composition can be included in one or more capsules or tablets.

In the present disclosure, 300 mg to 4000 mg of the Antrodia cinnamomea composition is administered to the subject per day, for example, 800 mg to 4000 mg, 1200 mg to 4000 mg, 1300 mg to 4000 mg, 1400 mg to 3800 mg, 1400 mg to 3500 mg, 1500 mg to 3500 mg, 1500 mg to 3300 mg, 1600 mg to 3300 mg, 1600 mg to 3000 mg, 1600 mg to 2800 mg, 1600 mg to 2500 mg, 1600 mg to 2300 mg or 1600 mg to 2000 mg of the Antrodia cinnamomea composition per day.

In the present disclosure, the frequency for taking the pharmaceutical composition or the Antrodia cinnamomea composition can be once to three times a day. In addition, the time for taking the pharmaceutical composition or the Antrodia cinnamomea composition is not particularly limited, and the pharmaceutical composition or the Antrodia cinnamomea composition can be taken before meal, after meal, before going to bed or other time points.

In the present disclosure, the pharmaceutical composition or the Antrodia cinnamomea composition is administered to the subject for at least 7 days to achieve the purpose of immune modulation.

In the present disclosure, the term “effective amount” refers to the amount of the composition which is required to confer the desired effect on the subject. Effective amounts vary, as recognized by those skilled in the art, depending on route of administration, excipient usage, and the possibility of co-usage with other therapeutic treatments such as use of other active agents.

In the present disclosure, the pharmaceutical composition may further comprise a pharmaceutically acceptable carrier, stabilizer, diluent, dispersant, thickener, excipient or a combination thereof.

In the present disclosure, the term “acceptable” means that the component must be compatible with the Antrodia cinnamomea composition (and preferably, capable of stabilizing the Antrodia cinnamomea composition) and not be deleterious to the subject to be administered.

Other novel features of the disclosure will become more apparent from the following detailed description when taken in conjunction with the accompanying drawings.

### BRIEF DESCRIPTION OF THE DRAWINGS

FIG. 1 is a diagram showing the profiling results of PD-L1⁺ B cells in the subjects.

FIG. 2 is a diagram showing the profiling results of PD-L1⁺ monocytes in the subjects.

FIG. 3 is a diagram showing the profiling results of MHC II⁺ dendritic cells in the subjects.

FIG. 4 is a diagram showing the profiling results of effector CD4 αβ T cells in the subjects.

FIG. 5 is a diagram showing the profiling results of effector CD8 αβ T cells in the subjects.

### DETAILED DESCRIPTION OF EMBODIMENT

Different embodiments of the present disclosure are provided in the following description. These embodiments are meant to explain the technical content of the present disclosure, but not meant to limit the scope of the present disclosure. A feature described in an embodiment may be applied to other embodiments by suitable modification, substitution, combination, or separation.

Preparation of Antrodia cinnamomea Composition

The Antrodia cinnamomea composition of the present disclosure is consisted of: 50 wt % to 99 wt % of solid-state cultivated Antrodia cinnamomea mycelium; and 1 wt % to 50 wt % of an ethanol/water extract of a fruiting body of Antrodia cinnamomea. The Antrodia cinnamomea mycelium is solid cultivated with whole grains (the inoculation ratio is 5-15% (v/w)). After cultivating at 18˜30° C. for 3-5 months, the solid-state cultivated Antrodia cinnamomea mycelium are harvested and processed to obtain Antrodia cinnamomea mycelium powders. In addition, the extract of the fruiting body of Antrodia cinnamomea is prepared by the following procedures. The fruiting body of Antrodia cinnamomea is harvest from the camphorata segment, the harvested fruiting body is dried, the dried fruiting body is extracted by water (50˜100° C.) and ethanol (35˜50° C.) in a ratio of 1:8 to 1:12 (W/V) for 1˜10 hours, the extraction process is repeated for 1 to 2 times, and the obtained extract is concentrated for the following use.

The obtained powders of the solid-state cultivated Antrodia cinnamomea mycelium and the water/ethanol extract of the fruiting body of Antrodia cinnamomea are mixed in a ratio of 99:1 to 1:1 to obtain the Antrodia cinnamomea composition. One combination is: 80 g of the powders of the solid-state cultivated Antrodia cinnamomea mycelium is mixed with 20 g of the water/ethanol extract of the fruiting body of Antrodia cinnamomea. Another combination is: 90 g of the powders of the solid-state cultivated Antrodia cinnamomea mycelium is mixed with 10 g of the water/ethanol extract of the fruiting body of Antrodia cinnamomea.

The Antrodia cinnamomea composition named “LEAC102 sample” used in the following embodiments is manufactured and provided by Taiwan Leader Biotech Corporation, which is the first combination mentioned above. After analyzing the components in the Antrodia cinnamomea composition, the active ingredients comprised in each grain of the Antrodia cinnamomea composition include: 4,7-dimethoxy-5-methyl-1,3-benzodioxole (2.9 mg˜17 mg), 4-hydroxy-2,3-dimethoxy-6-methyl-5-[3,7,11-trimethyl-2,6,10-dodecatriene]-2-cyclohexenone (0.35 mg˜21 mg), coenzyme Q3 (0.23 mg˜10 mg), Safrole C (0.24 mg˜9 mg), Zhankuic acid B (0.09 mg˜5 mg), Zhankuic acid H (0.25 mg˜13 mg), Zhankuic acid K (0.15 mg˜8 mg), Zhankuic acid A (0.005 mg˜2 mg), Zhankuic acid C (0.03 mg˜2 mg), dehydrosulfurenic acid (0.14 mg˜11 mg) and dehydroeburicoic acid (0.15 mg˜15 mg).

### Experimental Method

Healthy subjects were participated in the present embodiment. Flow cytometry was taken as an approach in evaluating immune regulatory responses for 4 times. In the present embodiment, all the subjects were orally up-taking LEAC102 sample twice daily for 3 months, and the intake amount was about 800˜1000 mg Antrodia cinnamomea composition each time. The associations of immune cells with indexes of immune modulatory function were analyzed by paired student t test analysis.

The reagents used in the present embodiment included: Ficoll-Paque PREMIUM (GE Healthcare cat#17-5442), Staining buffer (homemade reagent), eBioscience™ Foxp3/Transcription Factor Staining Buffer Set (eBioscience cat#00-5523-0), Pasteur Pipettes (Volac cat#D812), 1.5 ml eppendorf tubes (SSI cat#1260-00), 15 ml and 50 ml Plastic centrifuge tubes (Corning CentriStar cat#4307941/#430829), Thermo Scientific Nunc Serological Pipette (cat#170355/#170356/#170357), 96 well plate (V bottom) (for cell counting) (Basic Life cat#BL6001) and FACS™ Tubes (flow tube)(Corning cat#FLCON 352008).

Human PBMCs were isolated from buffy coats by Ficoll density gradient centrifugation technique (Ficoll-Paque™ PREMIUM; GE Healthcare, USA). After centrifugation, PBMCs were washed with 1×PBS and re-suspended with staining buffer (2% FBS+0.02% NaN₃ in 1×PBS) for cell surface marker staining or intracellular staining.

PBMCs were prepared from ficolled peripheral blood and stained for cell surface markers including Fluorescein isothiocyanate (FITC)-labeled anti-human T-cell receptor alpha/beta (TCRα/β), CD33; Phycoerythrin (PE)-labeled anti-human CD4, CD14, CD25, CD39, PD-L-1, PD-1; Coupled Dye (ECD)-labeled CD45; APC-labeled anti-human TCR gamma/delta (TCRγ/δ), CD11c; and AlexaFluor® 488APC-labeled anti-human CD25, PD-1. Data acquisition was performed on Navios flow cytometer (Beckman Coulter) and analyzed by Kaluza software version 2.1 (Beckman Coulter).

Statistical analysis was performed in-depth by comprehensively screening phenotypes of immune subsets and comparing the difference at kinetic time points. The data at kinetic time points were qualitatively and statistically analyzed by paired Student's T test.

### Results

Elucidation of Antrodia cinnamomea Composition on PD-L1⁺ Cell Subsets in PBMCs

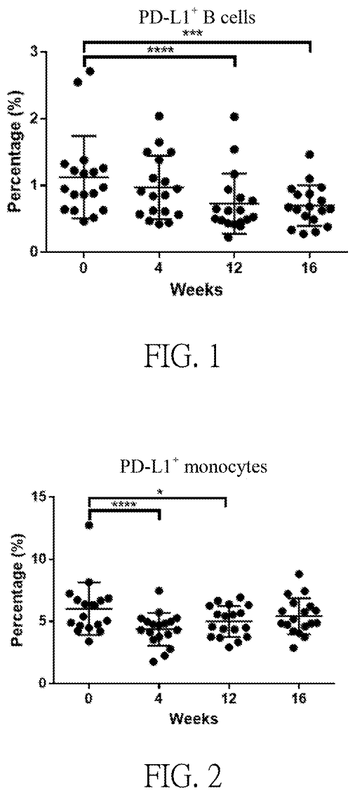

PD-1/PD-L1 axis plays an essential role in regulating immune balance. PD-L1 is the protein that expresses on normal and cancerous cells and delivers the negative signal to PD-1 expressing T cell to suppress T cell activation. To further confirm the effect of the Antrodia cinnamomea composition of the present embodiment on the regulation of the PD-L1/PD-1 axis, PBMCs of the subjects were collected and determined by flow cytometer and antibodies against specific epitopes, and analyzed by the aforesaid software (*, P<0.05, **, P<0.01, ***, P<0.001). The results are shown in FIG. 1 and FIG. 2, wherein FIG. 1 is a diagram showing the profiling results of PD-L1⁺ B cells in the subjects, and FIG. 2 is a diagram showing the profiling results of PD-L1⁺ monocytes in the subjects.

As shown in FIG. 1, at the 12^{th} and 16^{th} week, the expression of PD-L1⁺ B cells are significantly lower than that of the 0 week. As shown in FIG. 2, at the 4^{th} and 12^{th} week, the expression of PD-L1⁺ monocytes are significantly lower than that of the 0 week. These results indicate that the expression level of PD-L1 on B cells and monocytes was decreased. These data revealed that orally up-taking of the Antrodia cinnamomea composition may help block the interaction of PD-L1 and PD-1.

Elucidation of Antrodia cinnamomea Composition on MHC-II⁺ DC in PBMCs

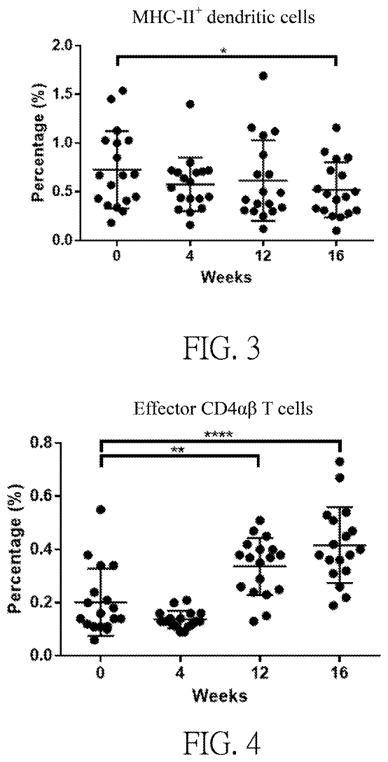

Dendritic cells (DCs) are potent antigen presenting and processing cells that help provide suitable antigens to both T cells and B cells. In Human DCs, all types of DCs demonstrate high levels of MHC class II related markers. To further confirm the effect of the Antrodia cinnamomea composition on the regulation of MHC-II positive cell subsets, PBMCs of the subjects were collected and determined by flow cytometer and antibodies against specific epitopes, and analyzed by the aforesaid software (*, P<0.05, **, P<0.01, ***, P<0.001). The results are shown in FIG. 3, which is a diagram showing the profiling results of MHC II⁺ dendritic cells in the subjects.

As shown in FIG. 3, at the 16^{th} week, the expression of MHC-II⁺ DCs are significantly lower than that of the 0 week. These data revealed that orally up-taking of the Antrodia cinnamomea composition may help pay tribute to suppressing cross-presentation activities in the subjects.

Elucidation of Antrodia cinnamomea Composition on Effector CD4 αβ T Cells and Effector CD8 αβ T Cells in PBMCs

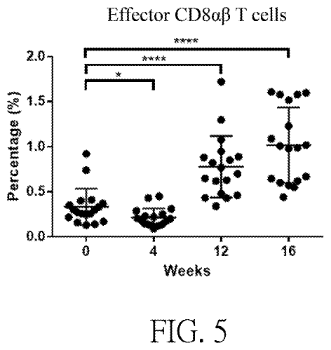

T cell is one of an essential immune subset against extracellular and intracellular pathogens. T cells also can distinguish into two major subgroups, CD4 and CD8 T cell, which indicated T helper cells and cytotoxic T cells, respectively. In addition, T cell becomes activated after encounters stimuli and up-regulates surface antigens expression. To confirm the effect of the Antrodia cinnamomea composition on the regulation of the effector CD4 αβ T cells and effector CD8 αβ T cells, PBMCs of the subjects were collected and determined by flow cytometer and antibodies against specific epitopes, and analyzed by the aforesaid software (*, P<0.05, **, P<0.01, ***, P<0.001). The results are shown in FIG. 4 and FIG. 5, which respectively are diagrams showing the profiling results of effector CD4 αβ T cells and effector CD8 αβ T cells in the subjects.

As shown in FIG. 4, at the 12^{th} and 16^{th} week, the expression of effector CD4αβ T cells in all the subjects are significantly higher than that of the 0 week. As shown in FIG. 5, at the 12^{th} and 16^{th} week, the expression of effector CD8αβ T cells in all the subjects are significantly higher than that of the 0 week. These results indicate the up-regulation of effector CD4 αβ T cells and effector CD8 αβ T cells expression. These data revealed that orally up-taking of the Antrodia cinnamomea composition have the potential to induce T cell activation.

The aforesaid results indicate that the Antrodia cinnamomea composition of the present disclosure has the effect of immune modulation by down-regulating the PD-L1⁺ B cells, the PD-L1⁺ monocytes and the MHC II⁺ dendritic cells and up-regulating the effector CD4 αβ T cells and the effector CD8 αβ T cells in healthy subjects. In particular, the Antrodia cinnamomea composition of the present disclosure may pay tribute to down-regulation of the PD-L1/PD-1 axis and up-regulation of the effector CD4 αβ T cells and the effector CD8 αβ T cells. Furthermore, the Antrodia cinnamomea composition of the present disclosure is effective in the modulation of the adaptive immunity. In the cell profiling results, not only the expressions of the MHC-II⁺ dendritic cells were significantly down-regulated, the expression levels of PD-L1⁺ monocytes were also decreased after 16 weeks of the administration of the Antrodia cinnamomea composition of the present disclosure. These discrete results showed that the Antrodia cinnamomea composition of the present disclosure may possess potential therapeutic effects in regulating the adaptive immune responses of the subjects.

Although the present disclosure has been explained in relation to its embodiment, it is to be understood that many other possible modifications and variations can be made without departing from the spirit and scope of the disclosure as hereinafter claimed.

What is claimed is:

1. A method for modulating an immune system in a subject in need thereof, comprising:
administering to a subject an effective amount of a pharmaceutical composition comprising an Antrodia cinnamomea composition, wherein the Antrodia cinnamomea composition comprises: 50 wt % to 100 wt % of Antrodia cinnamomea mycelium; and 0 wt % to 50 wt % of an extract of fruiting body of Antrodia cinnamomea.

2. The method of claim 1, wherein the Antrodia cinnamomea mycelium is solid-state cultivated Antrodia cinnamomea mycelium.

3. The method of claim 1, wherein the extract of the fruiting body of Antrodia cinnamomea is an ethanol extract of the fruiting body of Antrodia cinnamomea extracted with 50 wt % to 95 wt % of ethanol.

4. The method of claim 1, wherein PD-L1⁺ B cells in the subject are decreased.

5. The method of claim 4, wherein the PD-L1⁺ B cells in peripheral blood of the subject are decreased after administering the effective amount of the pharmaceutical composition, compared with the PD-L1⁺ B cells in the peripheral blood of the subject before administering the effective amount of the pharmaceutical composition.

6. The method of claim 5, wherein the PD-L1⁺ B cells in the peripheral blood are detected by flow cytometry, Enzyme-linked Immunospot Assay or immunohistochemistry.

7. The method of claim 1, wherein PD-L1⁺ monocytes in the subject are decreased.

8. The method of claim 7, wherein the PD-L1⁺ monocytes in peripheral blood of the subject are decreased after administering the effective amount of the pharmaceutical composition, compared with the PD-L1⁺ monocytes in the peripheral blood of the subject before administering the effective amount of the pharmaceutical composition.

9. The method of claim 8, wherein the PD-L1⁺ monocytes in the peripheral blood are detected by flow cytometry, Enzyme-linked Immunospot Assay or immunohistochemistry.

10. The method of claim 1, wherein MHC II⁺ dendritic cells in the subject are decreased.

11. The method of claim 10, wherein the MHC II⁺ dendritic cells in peripheral blood of the subject are decreased after administering the effective amount of the pharmaceutical composition, compared with the MHC II⁺ dendritic cells in the peripheral blood of the subject before administering the effective amount of the pharmaceutical composition.

12. The method of claim 11, wherein the MHC II⁺ dendritic cells in the peripheral blood are detected by flow cytometry, Enzyme-linked Immunospot Assay or immunohistochemistry.

13. The method of claim 1, wherein effector CD4 αβ T cells in the subject are increased.

14. The method of claim 13, wherein the effector CD4 αβ T cells in peripheral blood of the subject are increased after administering the effective amount of the pharmaceutical composition, compared with the effector CD4 αβ T cells in the peripheral blood of the subject before administering the effective amount of the pharmaceutical composition.

15. The method of claim 14, wherein the effector CD4 αβ T cells in the peripheral blood are detected by flow cytometry, Enzyme-linked Immunospot Assay or immunohistochemistry.

16. The method of claim 1, wherein effector CD8 αβ T cells in the subject are increased.

17. The method of claim 16, wherein the effector CD8 αβ T cells in peripheral blood of the subject are increased after administering the effective amount of the pharmaceutical composition, compared with the effector CD8 αβ T cells in the peripheral blood of the subject before administering the effective amount of the pharmaceutical composition.

18. The method of claim 17, wherein the effector CD8 αβ T cells in the peripheral blood are detected by flow cytometry, Enzyme-linked Immunospot Assay or immunohistochemistry.

19. The method of claim 1, wherein the pharmaceutical composition comprises 300 mg to 1500 mg of the Antrodia cinnamomea composition.

20. The method of claim 1, wherein 300 mg to 4000 mg of the Antrodia cinnamomea composition is administered to the subject per day.
